(19) United States
(12) Patent Application Publication
(10) Pub. No.: US 2011/0300092 A1
(43) Pub. Date: Dec. 8, 2011

(54) MASCARAS OBTAINED VIA MULTI-PHASE PRODUCT

(75) Inventors: Diane M. Kambach, Burlington, NJ (US); Sue Feng, Edison, NJ (US)

(73) Assignee: L'OREAL, Paris (FR)

(21) Appl. No.: 13/152,953

(22) Filed: Jun. 3, 2011

Related U.S. Application Data
(60) Provisional application No. 61/351,445, filed on Jun. 4, 2010.

(51) Int. Cl. A61K 8/92; A61Q 1/10

(52) U.S. Cl. 424/70.7; 132/218

(57) ABSTRACT

Disclosed is a multi-phase mascara product, including a first container having disposed therein a first phase comprising a wax, a second container having disposed therein a second phase comprising a film forming polymer, and a third container into which relative amounts of said first and second phases may be introduced, wherein upon mixing of the first and second phases, the third container comprises the wax, the film-forming polymer, a solvent and a thickener, wherein if the solvent and thickener are oil-soluble, they are present in the first phase, and if the solvent is aqueous and the thickener is water-soluble, they are present in the second phase. Also disclosed are methods of producing the mascara, by mixing relative amounts of the phases into a separate container. Further disclosed are methods of making up the eye lashes by application of the mascara.

### CROSS-REFERENCE TO RELATED APPLICATIONS

This application claims the benefit of the filing date of U.S. Provisional Patent Application No. 61/351,445 filed Jun. 4, 2010, the disclosure of which is hereby incorporated herein by reference.

### BACKGROUND OF THE INVENTION

The present invention generally relates to a novel multi-phase mascara product. More particularly the present invention relates to a multi-phase mascara product having a first phase containing a fluid mascara and a second phase containing ingredients for improving the comfort and application of the mascara.

Many mascaras have excellent volume, length, and/or wear properties. However, these mascaras do not provide a pleasant sensory experience during application. Such mascaras can be dry, have poor buildability, and/or give uneven coverage.

Moreover, conventional mascaras do not allow the consumer to alter the formula at the point of application. Thus, conventional mascaras provide the consumer with no ability to modify or tailor the properties of the mascara to suit their needs.

Therefore, it is desirable to provide a multi-phase mascara product having phases that are mixed just prior to application to provide improved comfort and application, while allowing the consumer flexibility in choosing the properties of the mascara composition.

### SUMMARY OF THE INVENTION

A first aspect of the present invention is directed to a multi-phase mascara product, e.g., in the form of a package or integral assembly, comprising a first container having disposed therein a first phase comprising a wax, a second container having disposed therein a second phase comprising a film forming polymer, and a third container into which relative amounts of the first and second phases may be introduced. Upon mixing of the first and second phases, the third container comprises the wax, the film-forming polymer, a solvent and a thickener. If the solvent and thickener are oil-soluble, they are present in the first phase, and if the solvent is aqueous and the thickener is water-soluble, they are present in the second phase.

Methods of producing the mascara, entailing mixing relative amounts of the phases into a separate container are also provided.

Methods of making up the eye lashes, entailing application of the mascara, are further provided.

It has been surprisingly discovered that the use of a multi-phase mascara product provides a pleasant sensory experience and improved application properties while maintaining the desirable volume, length, texture, and wear properties.

### BRIEF DESCRIPTION OF THE DRAWINGS

FIG. 1 depicts a representative embodiment of a two-phase a product package in accordance with certain aspects of the present invention.

FIGS. 2A-C depict various representative positions of the optional configuration lever 128 in relation to the aperture 114.

### DETAILED DESCRIPTION

Other than in the operating examples, or where otherwise indicated, all numbers expressing quantities of ingredients and/or reaction conditions are to be understood as being modified in all instances by the term “about,” meaning within 10% to 15% of the indicated number.

“Keratinous tissues”, as used herein, include but are not limited to, eyelashes, skin, hair and nails.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C. “Non-volatile”, as used herein, means having a flash point of greater than about 100° C.

As used herein, the expression “at least one”, “a” or “an” means one or more and thus includes individual components as well as mixtures/combinations.

“Volume,” as used herein, refers to the apparent build up of the composition on a keratinous tissue. In the example of a mascara, as volume increases each eyelash is defined and stands out to the eye, creating the desirable impression of longer and fuller lashes. A composition that produces greatly increased thickness is considered to have good volume properties. Volume may be evaluated by any method known in the art for evaluating such properties. For example, volume may be evaluated by a test involving first measuring the thickness of human eyelashes or hair. The composition is then applied to the eyelashes or hair. After a period of drying, the thickness of the eyelashes or hair is once again measured. The difference in volume from before and after treatment is then determined. Further, these characteristics may be evaluated with respect to other compositions, such as commercially available compositions.

An aspect of the present invention is directed to a multi-phase mascara product. A first phase of the multi-phase mascara product includes, in a first container, a wax or other suitable structuring agent, and to the extent they are present, an oil-based solvent, e.g., a hydrocarbon solvent, and an oil-soluble thickener. A second phase of the mascara product, in a second container, includes a film-forming polymer and to the extent they are present, an aqueous solvent and a water-soluble thickener. The multi-phase mascara product further includes a third container into which relative amounts of the first and second phases may be introduced, wherein upon mixing of the first and second phases, the third container comprises the wax, the film-forming polymer, a solvent and a thickener. If the solvent and thickener are oil-soluble, they are present in the first phase, and if the solvent is aqueous and the thickener is water-soluble, they are present in the second phase.

### Wax

According to the present invention, multi-phase mascara products comprising a wax are provided. For the purposes of the present invention, the term “wax” means a lipophilic fatty compound that is solid at room temperature about (25° C.) and atmospheric pressure (760 mmHg, i.e., 105 Pa), which undergoes a reversible solid/liquid change of state and which has a melting point of greater than 30° C. and in some embodiments, greater than about 55° C. up to about 120° C. or even as high as about 200° C. For the purposes of the invention, the waxes are those generally used in cosmetics and dermatology. A variety of waxes may be useful, including waxes of animal origin, waxes of plant origin, waxes of mineral origin and waxes of synthetic origin. Representative examples of suitable waxes of animal origin include beeswaxes, lanolin waxes and Chinese insect waxes. Representative examples of suitable waxes of plant origin include rice waxes, carnauba wax, candelilla wax, ouricurry wax, cork fibre waxes, sugar cane waxes, Japan waxes, sumach wax and cotton wax. Representative examples of suitable waxes of mineral origin include paraffins, microcrystalline waxes, montan waxes and ozokerites. Representative examples of suitable waxes of synthetic origin include polyolefin waxes, e.g., polyethylene waxes, waxes obtained by Fischer-Tropsch synthesis, waxy copolymers and their esters, and silicone and fluoro waxes. Alternatively, hydrogenated oils of animal or plant origin may be used. Examples include hydrogenated jojoba waxes and hydrogenated oils which are obtained by catalytic hydrogenation of fats composed of a C8-C32 linear or nonlinear fatty chain, hydrogenated sunflower oil, hydrogenated castor oil, hydrogenated copra oil, hydrogenated lanolin and hydrogenated palm oils. The wax may be self-emulsifying. Commercially available self-emulsifying waxes that may be useful in the practice of the present invention include the following: PEG-20 sorbitan beeswax (Atlas G-1726, Uniqema; Nikkol GBW-125, Nikko), PEG-6 beeswax (ESTOL 375, Uniqema), PEG-8 beeswax (Apifil, Gattefosse), PEG-12 beeswax and PEG-12 carnauba wax. In some embodiments, the compositions contain at least two or at least three waxes. In embodiments of the present invention, the wax is present in an amount generally ranging from about 0.1% to about 40%, and in some embodiments from about 0.5% to about 20%, or from about 1% to about 10%, by weight, relative to the total weight of the mascara.

### Solvent

According to the present invention, multi-phase mascara products comprising a solvent are provided. The solvent may be non-aqueous or aqueous-based (aqueous), polar or non-polar and/or volatile or non-volatile. Representative examples of suitable volatile solvents include non-polar volatile hydrocarbon-based oils (which as used herein, refers to oil containing only hydrogen and carbon atoms), silicone oils (optionally comprising alkyl or alkoxy groups that are pendant or at the end of a silicone chain), and fluoro oils. Suitable hydrocarbon-based oils include isoparaffins, i.e., branched alkanes containing 8-16 carbon atoms, such as isododecane (also known as 2,2,4,4,6-pentamethylheptane), and petroleum distillates. Suitable silicone oils may include linear or cyclic silicones containing from 2 to 7 silicon atoms, these silicones optionally comprising alkyl or alkoxy groups containing from 1 to 10 carbon atoms. Examples include octamethylcyclotetrasiloxane, decamethylcyclopentasiloxane, hexadecamethylcyclohexasiloxane, heptamethylhexyltrisiloxane and heptamethyloctyltrisiloxane, and mixtures thereof. Mixtures of these solvents may be used. Polar volatile solvents may also be used, examples of which include C2 to C5 alcohols, such as ethanol, ethyl 3-ethoxypropionate and isohexyl neopentanoate.

In an embodiment of the present invention, it is preferred to employ a volatile solvent. The volatile solvent is present in an amount generally ranging from about 0.1% to about 90%, and in some embodiments, from about 5% to about 80%, and in other embodiments, from about 10% to about 70%, by weight, based on the total weight of the mascara.

Representative examples of suitable non-polar non-volatile solvents include polyalphaolefins, which include ethylene derivatives oligomerized into even-numbered carbon polyalphaolefins e.g., C6-C14 olefins such as polydecene and polymers of C6, C8, C12 and C14 olefins. The polyolefins may have a molecular weight (MW) generally ranging from about 280 to about 11,500, and a viscosity (CPs at about 20° C.) generally ranging from about 7 to about 32,500. They may also be hydrogenated. In some embodiments, the non-volatile solvent includes PureSyn™ 2 (MW about 283), 4 (MW about 432), 6 (MW about 570), 8 (MW about 611), 150 (MW about 3980) and 300 (MW about 4870) (INCI name: hydrogenated polydecene). The viscosity of these polymers is about 8, about 33, about 64, about 103, about 4179 and about 8400, respectively.) PureSyn™ 100 (MW about 2939, viscosity about 3900, INCI name:hydrogenated C6-14 olefin polymers) and PureSyn™ 1000 (MW about 11,500, viscosity about 32,400, INCI name: polydecene) may also be useful. The PureSyn™ products are available from Exxon Chemicals.

In embodiments of the present invention, the non-volatile solvent is present in an amount generally ranging from about 0.1% to about 70%, and in some embodiments, about 0.5% to about 40%, and in other embodiments, 1% to about 25%, by weight, based on the total weight of the mascara.

The inventive compositions may contain any other cosmetically or dermatologically acceptable and, in general, physiologically acceptable oil, such as carbon-based, hydrocarbon-based, fluoro and/or silicone oils, of mineral, animal, plant or synthetic origin, alone or as a mixture. These ingredients, along with any non-polar solvents, would constitute a liquid fatty phase of the mascara composition.

The solvent may or may not include water. Mascara compositions that are washable contain water. On the other hand, mascara compositions that are waterproof, may or may not contain water. Generally, water content of washable mascara compositions ranges from about 20 to about 80%, by weight, and in some embodiments from about 30 to about 60%, by weight, based on the total weight of the mascara. In contrast, water content of waterproof mascara compositions generally ranges from zero or greater than zero to about 60%, by weight, and in some embodiments from zero or greater than zero to about 35%, by weight, based on the total weight of the mascara. One or more water-miscible solvents (having a miscibility in water of greater than 50% by weight at about 25° C.) may also be present. Examples include lower, i.e., C₁-C₅ monoalcohols such as ethanol and isopropanol, C₂-C₈ glycols containing such as propylene glycol, ethylene glycol, butylene glycol, dipropylene glycol, and pentylene glycol, C₃-C₄ ketones, and C₂-C₄ aldehydes.

Thus, the compositions in an embodiment of the present invention contain a mixture of solvents, such as water and butylene glycol or propylene glycol.

### Emulsifier

In those embodiments wherein the mascara composition contains water, the compositions of the invention also contain an emulsifier. Emulsifiers typically employed in the compositions of the present invention include anionic, nonionic and cationic emulsifiers. Representative examples of suitable emulsifiers include as nonionic emulsifiers, fatty acids, fatty alcohols, polyethoxylated fatty alcohols or polyglycerolated fatty alcohols, such as polyethoxylated stearyl alcohols or cetylstearyl alcohols, esters of fatty acid and sucrose, and glucose alkyl esters, in particular polyoxyethylenated C1-C6 alkyl glucose fatty esters, and as anionic emulsifiers, C16-C30 fatty acids neutralized by amines, ammonia or the alkali metal salts thereof. Examples of cationic emulsifiers include quaternary amines, amine oxides and amines, e.g., alkyl amines, alkyl imidazolines, ethoxylated amines, quaternary compounds, and quaternized esters. In addition, to the roles in stabilizing phases in an emulsion, cationic emulsifiers may also provide a conditioning effect.

The mascara product of the present invention may also contain a silicone emulsifying surfactant. Representative examples of suitable silicone emulsifying surfactants include polydimethylsiloxanes comprising oxyethylenated and/or oxypropylenated groups. Use is preferably made of those comprising an oxyethylenated and/or oxypropylenated chain as shown below: —(C₂H₄O)ₓ—(C₃H₆O)_{y}—H where x can range from zero to 100 and y can range from zero to 100, however x and y cannot both represent 0. According to an embodiment of the invention, these silicone surfactants comprise both oxyethylenated groups and oxypropylenated groups, for example from 2 to 50 oxyethylenated groups and from 2 to 50 oxypropylenated groups, each range of 2-50 including 5, 10, 20, 30, and 40, as well as the subrange 16-20. The silicone surfactants used in the composition of the invention can be chosen from, for example, dimethicone copolyols and alkyl dimethicone copolyols. The dimethicone copolyols that can be used in the composition of the invention include in particular those comprising from 2 to 50 oxyethylenated groups and from 2 to 50 oxypropylenated groups, for instance that comprising 17 oxyethylenated groups and 18 oxypropylenated groups (PEG/PPG-17/18 dimethicone).

An emulsifier, if present, is in amounts generally ranging from about 1 to about 30%, and in some embodiments from about 3% to about 15%, by weight, based on the total weight of the mascara.

### Film-Forming Polymer

According to the present invention, embodiments of a multi-phase mascara product comprising a film-forming polymer are provided. The film-forming polymer can be selected from keratin derivatives, such as keratin hydrolysates and sulphonic keratins; anionic, cationic, amphoteric or nonionic derivatives of chitin or chitosan; cellulose derivatives such as hydroxyethylcellulose, hydropropylcellulose, methylcellulose, ethylhydroxyethylcellulose, carboxymethylcellulose, and quaternized derivatives of cellulose; acrylic and methacrylic polymers or copolymers, such as polyacrylates or polymethacrylates, and salts (e.g., sodium salts) thereof; polyvinylpyrrolidones (PVP) and vinyl copolymers, such as methyl vinyl ether-maleic anhydride copolymers, or vinyl acetate-crotonic acid copolymer; water-dispersible anionic polyesteramide and/or polyester polymers comprising monomers bearing a functional group —SO3M, in which M represents a hydrogen atom, an ammonium ion NH4+ or a metal ion, such as, for example, an Na⁺, Li⁺, K⁺, Mg²⁺, Ca²⁺Cu²⁺, Fe²⁺ or Fe³⁺ ion. Specific examples of the polymers are described in U.S. Pat. Nos. 3,734,874, 4,233,196 and 4,304,901. Polyurethane polymers, especially anionic, cationic, nonionic or amphoteric polyurethanes, acrylic polyurethanes, polyvinylpyrrolidone polyurethanes, polyester polyurethanes, polyether polyurethanes, polyureas, polyurea/polyurethanes, and mixtures thereof; and polymers of natural origin, modified if desired, such as gum arabic, guar gum, xanthan derivatives, karaya gum; alginates and carragheenates; glycoaminoglycans, hyaluronic acid and its derivatives; shellac, sandarac gum, dammars, elemis and copals, are also useful. Mention may also be made of quaternized polymers (which are typically cationic polymers, but may also include amphoteric and zwitterionic polymers) such as polyquaternium-4, polyquaternium-6, polyquaternium-7, polyquaternium-8, polyquaternium-9, polyquaternium-10, polyquaternium-22, polyquaternium-32, polyquaternium-39, polyquaternium-44 and polyquaternium-47.

In some embodiments, film forming polymer is a polyurethane/poly(meth)acrylate graft copolymer. These may also be referred to as an interpenetrated polymer network (IPN) of a polyurethane and a poly(meth)acrylate. As used herein, the expression “interpenetrated polymer network” refers to a blend of two interlaced polymers, obtained by simultaneous polymerization and/or crosslinking of two types of monomer, the blend obtained having a single glass transition temperature. Such IPNs are especially those that are commercially available from the company Air Products under the name Hybridur. An IPN that is particularly preferred is in the form of an aqueous dispersion of particles e.g., with a weight-average size of between 90 and 110 nm and a number-average size of about 80 nm. This IPN preferably has a glass transition temperature, Tg, ranging from about −60° C. to +100° C. An IPN of this type is available from Air Products under the trade name Hybridur 875 (INCI name: POLYURETHANE-2 (and) POLYMETHYL METHACRYLATE). Polyurethane/poly(meth)acrylics available from Air Products under the names Hybridur X-01602 and X 18693-21 are also disclosed in U.S. Publication Nos. 2003/0215476 and 2004/0136937.

The IPNs of the present invention are polyurethane/poly(meth)acrylate graft copolymers having the following general structure:

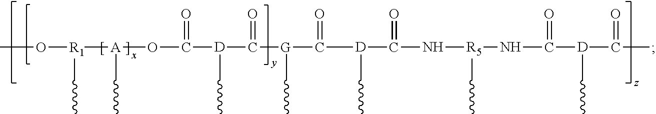

wherein

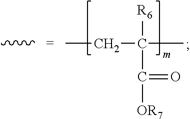

wherein

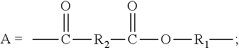

wherein D=—NH—R₃NH—;
and wherein

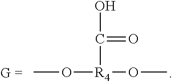

In the formula, R1, R2, R3, R4, R5 and R7 each independently represents an aliphatic hydrocarbon; m represents zero or a positive integer; R6 represents hydrogen or methyl; and x, y and z each independently represents a positive integer. The graft copolymers may be provided in the form of aqueous dispersions. The graft copolymers may be added to the other components of the mascara in powdered form as well.

In some embodiments, the film forming polymer includes an acrylates copolymer. Acrylates copolymers useful in the present invention are more generally referred to in the art as acrylic film-forming dispersions as they are commercially available in the form of liquid dispersions or emulsions. Suitable acrylates copolymers for use in the present invention include ethyl acrylates/methyl methacrylates copolymer emulsion (chemical name) (INCI name:water (and) acrylates copolymer), which is commercially available from Kobo Products, Inc. (South Plainfield, N.J.) and Daito Kasei Kogyo Co., Ltd., under the trade name Daitosol AD. This product is sold in the form of an emulsion that contains water, ethyl acrylates/methyl methacrylates copolymer, sodium dehydroacetate, and Laureth-20 (lauryl alcohol and oxirane). See, United States Patent Application Publication No. 20060134043 A1. Another suitable acrylates copolymer is ethyl methacrylates/N-butyl acrylates/2-methylhexyl acrylates copolymer emulsion (chemical name) (INCI name:water (and) acrylates/ethylhexyl acrylates copolymer), which is also commercially available from Kobo Products, Inc. and Daito Kasei Kogyo Co., Ltd., under the trade name Daitosol SJ. This product is sold in the form of an emulsion that contains water, ethyl methacrylates/N-butyl acrylates/2-methylhexyl acrylates copolymer, and Laureth-20. Other yet other acrylates copolymers include an alkyl (meth)acrylates copolymer emulsion (INCI name: acrylates copolymer), which is commercially available from Nippon LSC Ltd., under the trade name Yodosol GH34F; acrylates/ammonium methacrylates copolymer (INCI name) (CAS No. 25212-88-8), commercially available from Ganz Chemical under the tradename ULTRASOL, the chemical composition of which includes, in addition to the copolymer, water, zinc oxide, sodium lauryl sulfate, and methylparaben; a styrene/acrylates copolymer emulsion (INCI name), which is commercially available from Nippon LSC Ltd., under the tradename Yodosol GH41F; a styrene/acrylates copolymer emulsion (INCI name), and which is commercially available from BASF under the tradename Joncryl 77 (which contains the copolymer in the form of an ammonia salt, along with water and polypropylene glycol); a Polyacrylates-21 (and) acrylates/dimethylaminoethyl methacrylates copolymer (INCI name), commercially available from Interpolymer under the tradename Syntran 5100, the chemical composition of which includes, in addition to water and the two acrylates copolymers having CAS Nos. 68541-61-7 and 67380-24-9 respectively, ethoxylated secondary alcohol (CAS No. 84133-50-6) and sodium laurylpolyethoxyethanol sulfate (CAS No. 68891-38-3); a styrene/acrylates/ammonium methacrylates copolymer (and) butylene glycol (and) sodium Laureth-12 sulfate (INCI name), commercially available from Interpolymer under the tradename Syntran 5760 as a 40% aqueous dispersion; and a polyurethane-10 and PEG-12 dimethicone alcohol copolymer emulsion (INCI name), commercially available from Nippon LSC under the tradename Yodosol PUD (which also includes ethanol, 2-phenoyl-ethanol, and water in the emulsion).

In some embodiments, a tacky film forming polymer is present in the mascara composition. The selection of such polymer depends on the nature of the solvent system employed in formulating the mascara. Examples of tacky film formers soluble or dispersible in water include polyvinyl alcohol (available, for example, from Air Products in different hydrolysis grades under the tradename AIRVOL), polyvinyl acetates (such as FULATEX® sold by H.B. Fuller Co.), vinylpyrrolidone/acrylates/lauryl methacrylate copolymer (such as STYLEZE 2000 sold by ISP), acrylates/C1-2 succinates/hydroxyacrylates copolymer (such as ALLIANZ LT-120 sold by ISP), PVP/DMAPA acrylates copolymer (such as STYLEZE CC-10 sold by ISP), cellulose acetate phthalate aqueous dispersion (such as AQUACOAT CPD sold by FMC Corp.), and crosslinked poly(2-ethylhexyl acrylate) in water (such as GEL-TAC 100 series sold by API). Examples of tacky film formers soluble in oil include hydrogenated polyisobutenes (available from, for example, Collaborative Laboratories, East Setauket, N.Y., under the name POLYSYNLANE), adipic acid/diethylene glycol/glycerin crosspolymers (such as that sold as LEXOREZ 100 by Inolex), polyethylenes, polyvinyl laurates, and synthetic terpene based resins (such as PICCOLYTE A115 and C115, sold by Hercules).

In embodiments of the present invention, the film-forming polymer is present in an amount generally ranging from about 0.01 to about 20%, in other embodiments from about 0.5% to about 15% by weight, and in yet other embodiments from about 1% to about 5%, by weight, based on the total weight of the mascara.

### Fibers

According to the present invention, embodiments of a multi-phase mascara product comprising fibers are provided. Fibers provide enhanced lengthening effect. The fibers useful in the present invention may be chosen from natural and synthetic fibers. Representative examples of suitable natural fibers include cotton, silk, wool, and other keratin fibers. Representative examples of suitable synthetic fibers include polyester, rayon, nylon and other polyamide fibers, such as poly toluoylene co trimellic amide imide fibers. In embodiments of the present invention, fibers are present in an amount generally ranging from about 0.01% to about 10% by weight of the mascara.

### Thickener

Viscosity may be adjusted by adding a thickener or an agent useful for gelling a liquid phase. Both aqueous-based and oil-based ingredients may be employed. Representative examples are described in U.S. Patent Publications 2003/0215413, 2005/0065046 and 2002/0028226. Oil-based gelling agents may be chosen from gelling agents in polymeric form and gelling agents in mineral form. The gelling agent may be selected from the group consisting of agents that gel via chemical reticulation and agents that gel via physical reticulation. Modified clays may be used as gelling agents, examples of which include hectorites modified with an ammonium chloride of a C10 to C22 fatty acid, such as hectorite modified with distearyldimethylammonium chloride, also known as quaternium-18 bentonite, such as the products sold or made under the names Bentone 34 by the company Rheox, Claytone XL, Claytone 34 and Claytone 40 sold or made by the company Southern Clay, the modified clays known under the name quaternium-18 benzalkonium bentonites and sold or made under the names Claytone HT, Claytone GR and Claytone PS by the company Southern Clay, the clays modified with stearyldimethylbenzoylammonium chloride, known as stearalkonium bentonites, such as the products sold or made under the names Claytone APA and Claytone AF by the company Southern Clay, and Baragel 24 sold or made by the company Rheox. Other mineral gelling agents include silica, such as fumed silica. The fumed silica may have a particle size ranging from about 5 nm to 200 nm.

Water-soluble thickeners or gelling agents that may be used include polyvinylpyrrolidone (PVP), polyvinyl alcohol, crosslinked acrylates (e.g. Carbopol 982), hydrophobically-modified acrylates (e.g. Carbopol 1382); polyacrylamides such as, for example, the crosslinked copolymers sold under the names Sepigel 305 (CTFA name: polyacrylamide/C13-C14 isoparaffin/Laureth 7) or Simulgel 600 (CTFA name: acrylamide/sodium acryloyldimethyltaurate copolymer/isohexadecane/polysorbate 80) by SEPPIC; 2-acrylamido-2-methylpropanesulphonic acid polymers and copolymers, that are optionally crosslinked and/or neutralized; cellulose derivatives such as hydroxyethylcellulose, sodium carboxymethylcellulose, hydroxypropyl methylcellulose, hydroxypropyl cellulose, ethyl cellulose and hydroxymethyl cellulose; polysaccharides and gums, e.g., natural gums such as xanthan gum, sclerotium, carrageenan and pectin; polysaccharide resins such as starch and its derivatives, hyaluronic acid and its salts, clays, and, in particular, montmorillonites, hectorites, bentonites, and laponites, crosslinked polyacrylic acids, such as the “Carbopol” products from the company Goodrich, the polyglyceryl (meth)acrylates polymers sold under the names “Hispagel” or “Lubragel” by the companies Hispano Quimica or Guardian, crosslinked acrylamide polymers and copolymers, such as those sold under the names “PAS 5161” or “Bozepol C” by the company Hoechst, “Sepigel 305” by the company SEPPIC, crosslinked methacryloyloxyethyltrimethylammonium chloride homopolymers sold under the name “Salcare SC95” by the company Allied Colloid, and associative polymers and, in particular associative polyurethanes.

In embodiments of the present invention, the thickener is present in an amount generally ranging from about 0.01 to about 20%, in other embodiments from about 0.5% to about 15% by weight, and in yet other embodiments from about 1% to about 5%, by weight, based on the total weight of the mascara.

### Optional Ingredients

An optional ingredient may be present in one or both of the first and second phases based on its compatibility with the other ingredients in the phase.

### Moisturizer

Mascara compositions of the present invention may also contain a moisturizer. Examples include sodium lactate, mannitol, amino acids, hyaluronic acid, lanolin, urea, petroleum jelly and mixtures thereof. Other examples include polyols such as glycerin, diglycerin, triglycerin, polyglycerin, polyethylene glycol, ethylene glycol, diethylene glycol, triethylene glycol, propylene glycol, dipropylene glycol, hexylene glycol, 1,3-butylene glycol, 1,4-butylene glycol and sorbitol. A moisturizer, if present, is in amounts generally ranging from about 0.1% to about 20%, and in some embodiments, from about 0.5% to about 15%, by weight, based on the total weight of the mascara.

### Colorant

The inventive mascara compositions may also contain a colorant, e.g., pigment. Pigments may be chosen from the organic and/or mineral pigments known in the art. These pigments may be in the form of pigmentary powder or paste. They may be coated or uncoated.

The pigments may be chosen, for example, from mineral pigments, organic pigments, lakes, pigments with special effects such as nacres or glitter flakes, and mixtures thereof.

The pigment may be chosen from natural pigments. Natural pigments may be chosen from, for example, iron oxides, mica (e.g., mica sericite), chromium oxides, manganese violet, ultramarine blue, titanium dioxide, chromium hydrate and ferric blue.

The pigment may be an organic pigment. The organic pigment may be chosen from nitroso, nitro, azo, xanthene, quinoline, anthraquinone, phthalocyanin, metal-complex, isoindolinone, isoindoline, quinacridone, perinone, perylene, diketopyrrolopyrrole, thioindigo, dioxazine, triphenylmethane and quinophthalone compounds.

White or colored organic pigments may be chosen from carmine, carbon black, aniline black, melanin, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenolic derivatives as described in patent FR 2 679 771.

Examples that may also be mentioned include pigmentary pastes of organic pigments, such as the product sold by the company Hoechst under the names: Jaune Cosmenyl IOG: Pigment Yellow 3 (CI 11710); Jaune Cosmenyl G: Pigment Yellow 1 (CI 11680); Orange Cosmenyl GR: Pigment Orange 43 (CI 71105); Rouge Cosmenyl R: Pigment Red 4 (CI 12085); Carmine Cosmenyl FB: Pigment Red 5 (CI 12490); Violet Cosmenyl RL: Pigment Violet 23 (CI 51319); Bleu Cosmenyl A2R: Pigment Blue 15.1 (CI 74160); Vert Cosmenyl GG: Pigment Green 7 (CI 74260); Noir Cosmenyl R: Pigment Black 7 (CI 77266).

The pigments may also be in the form of composite pigments as described in patent EP 1 184 426. These composite pigments may be compounds of particles comprising a mineral core, at least one binder for ensuring the binding of the organic pigments to the core, and at least one organic pigment at least partially covering the core.

The organic pigment may also be a lake. The term “lake” means insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium.

Among the dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 0 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a non-uniform colored appearance (characterized by a certain shade, a certain vivacity and a certain lightness) that changes as a function of the conditions of observation (light, temperature, observation angles, etc.). They thus contrast with white or colored pigments that afford a standard uniform opaque, semi-transparent or transparent shade.

Several types of pigment with special effects exist: those with a low refractive index, such as fluorescent, photochromic or thermochromic pigments, and those with a high refractive index, such as nacres or glitter flakes.

Examples of pigments with special effects that may be mentioned include nacreous pigments such as white nacreous pigments such as mica coated with titanium or with bismuth oxychloride, colored nacreous pigments such as titanium mica with iron oxides, titanium mica with ferric blue or with chromium oxide, titanium mica with an organic pigment of the abovementioned type, and also nacreous pigments based on bismuth oxychloride. Nacreous pigments that may be mentioned include the Cellini nacres sold by Engelhard (mica-TiO2-lake), Prestige sold by Eckart (mica-TiO₂), Prestige Bronze sold by Eckart (mica-Fe₂O₃), and Colorona sold by Merck (mica-TiO₂—Fe₂O₂).

In addition to nacres on a mica support, multilayer pigments based on synthetic substrates such as alumina, silica, sodium calcium borosilicate or calcium aluminium borosilicate, and aluminium, may be envisaged.

Mention may also be made of pigments with an interference effect that are not fixed onto a substrate, for instance liquid crystals (Helicones HC from Wacker), holographic interference flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

Quantum dots are luminescent semiconductive nanoparticles capable of emitting, under light excitation, irradiation with a wavelength ranging from 400 nm to 700 nm. These nanoparticles are known from the literature. They may be manufactured in particular according to the processes described, for example, in U.S. Pat. No. 6,225,198 or U.S. Pat. No. 5,990,479, in the publications cited therein, and also in the following publications: Dabboussi B. O. et al. “(CdSe)ZnS core-shell quantum dots: synthesis and characterization of a size series of highly luminescent nanocrystallites” Journal of Physical Chemistry B, vol. 101, 1997, pp. 9463-9475 and Peng, Xiaogang et al. “Epitaxial growth of highly luminescent CdSe/CdS core/shell nanocrystals with photostability and electronic accessibility”, Journal of the American Chemical Society, vol. 119, No. 30, pp. 7019-7029.

The variety of pigments that may be used makes it possible to obtain a wide range of colors, and also particular optical effects such as metallic effects or interference effects.

A colorant, such as a pigment, if present, is in amounts generally ranging from about 0.01% to about 50%, based on the total weight of the mascara. In certain embodiments, the colorant, if present, is in an amount of from about 1% to about 45%, in other embodiments from about 10% to about 40%, and in yet other embodiments from about 30% to about 40%, by weight, based on the total weight of the mascara. In particular embodiments, the colorant, if present, is in an amount of about 35%, by weight, based on the total weight of the mascara.

### Dispersion Enhancing Agent

The mascara compositions of the present invention may also contain dispersion enhancing agents such as polysaccharide resins, e.g., KM 13, available from KAMA International Corp. (Duluth, Ga.). Dispersion enhancing agents are especially preferred in pigmented products. A dispersion enhancing agent, if present, is in amounts generally ranging from about 0.1% to about 5% by weight of the mascara.

### Fillers

Fillers, powders and mothers-of-pearl may also be present, typically to modify the texture of the composition and the matteness/gloss effect. Fillers should be understood to mean lamellar or non-lamellar, inorganic or synthetic, colorless or white particles. Mothers-of-pearl should be understood to mean iridescent particles produced especially by certain mollusks in their shell or else synthesized. Representative examples of these ingredients include mica, silica, kaolin, iron oxides, titanium dioxide, polyamide powders, polyamide powders, for instance Nylon® (Orgasol from Atochem), poly-alanine powders, polyethylene powders, tetrafluoroethylene polymer powders, for instance Teflon®, starch, boron nitride, hollow polymer microspheres such as those of polyvinylidene chloride/acrylonitrile, for instance Expancel® (Nobel Industrie), acrylic powders such as Polytrap® (Dow Corning), polymethyl methacrylates particles and silicone resin microbeads (for example Tospearls® from Toshiba), magnesium hydrocarbonate, hydroxyapatite, hollow silica microspheres (Silica Beads® from Maprecos), and glass and ceramic microcapsules. Filler(s), if present, are in amounts generally ranging from about 0.1% to about 25%, and in some embodiments from about 1% to about 20% by weight of the total weight of the mascara.

### Preservative

Representative examples of preservatives include alkyl para-hydroxybenzoates, wherein the alkyl radical has from 1, 2, 3, 4, 5 or 6 carbon atoms and preferably from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben), and phenoxyethanol. Mixtures of preservatives are also useful, e.g., the mixture of methylparaben, ethylparaben, propylparaben and butylparaben sold under the name Nipastat by Nipa, the mixture of phenoxyethanol, methylparaben, ethylparaben, propylparaben and butylparaben, also sold by Nipa under the name Phenonip, and the mixture of phenoxyethanol, methylparaben, isopropylparaben, isobutylparaben and butylparaben, sold by ISP under the tradename Liquapar Optima. A preservative, if present, is in amounts generally ranging from about 0.01% to about 15% by weight of the mascara.

The compositions of the present invention may further contain at least one further (e.g., cosmetically or dermatologically acceptable) ingredient, including additives and adjuvants, including, for example, gelling activators, humectants, polymers (other than the film-forming polymer or thickener), anti-foam agents (e.g., simethicone, which is a fluid composition containing polydimethylsiloxane and silica), sunscreen agents (e.g., inorganic sunscreen agent, such as titanium dioxide and zinc oxide and organic sunscreen agents, such as octocrylene, ethylhexyl methoxycinnamate, and avobenzone), chelators (such as EDTA and salts thereof, particularly sodium and potassium salts), antioxidants (e.g., BHT, tocopherol), essential oils, fragrances, and cosmetically active agents and dermatological active agents such as, for example, anti-inflammatory agents, vitamins, and trace elements. These ingredients may be soluble or dispersible in whatever phase or phases is/are present in the cosmetic composition (i.e., aqueous and/or fatty phase).

### DETAILED DESCRIPTION OF THE DRAWINGS

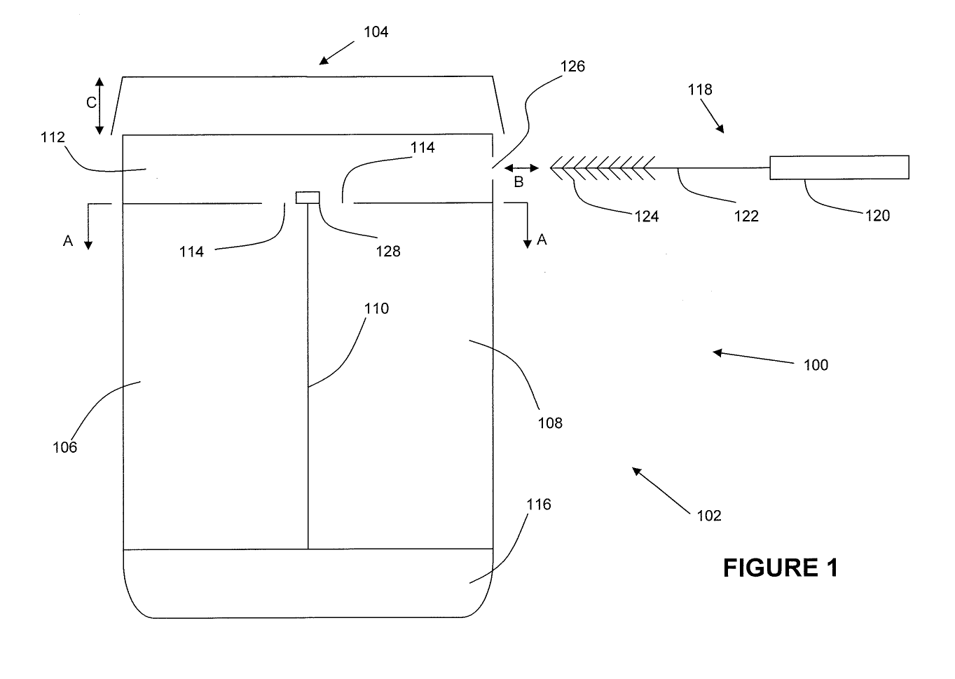

As previously discussed, the inventive mascara product may be stored and packaged for commercial use within a product package. FIG. 1 depicts a representative embodiment of such a product package in accordance with certain aspects of the present invention.

As shown in FIG. 1, the product package 100 may include a main body 102 and a cap 104. In order to separately house the inventive phases intended to form the mascara product, the main body 102 may be comprised of a first compartment 106 and a second compartment 108, where the first compartment houses the first phase and the second compartment houses the second phase. As an exemplary arrangement, the first phase may comprise a solvent and a wax and the second phase may comprise a film forming polymer, fibers, and/or a thickener.

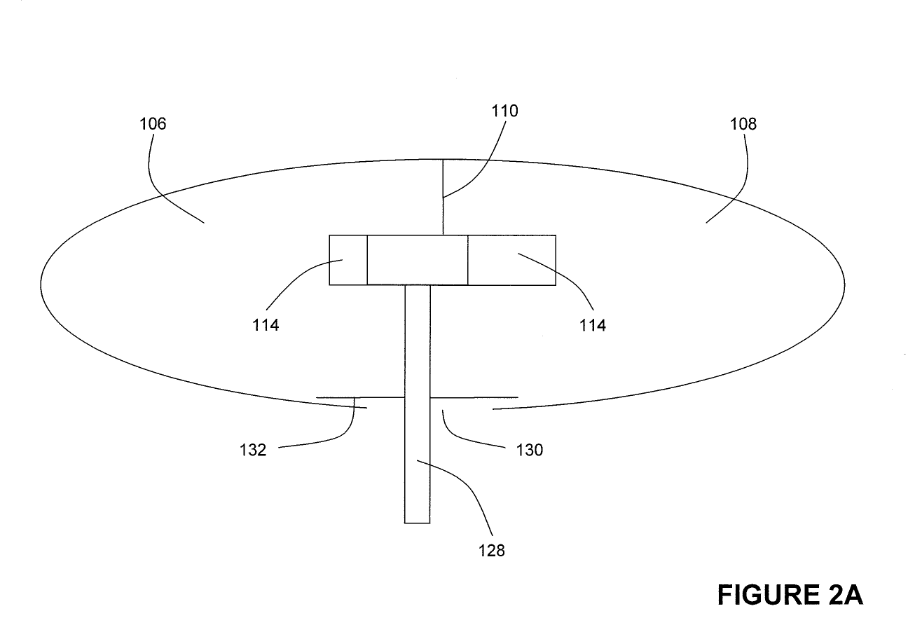

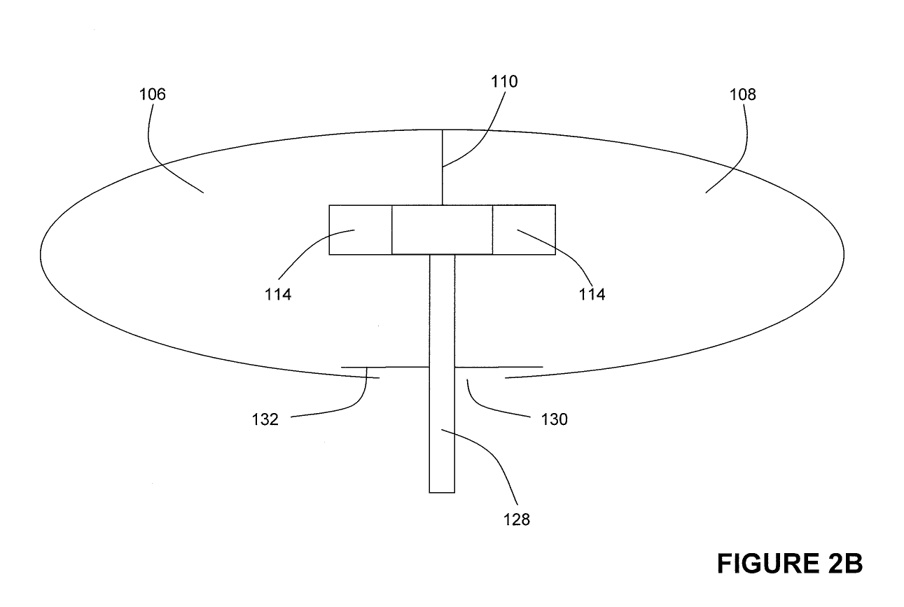

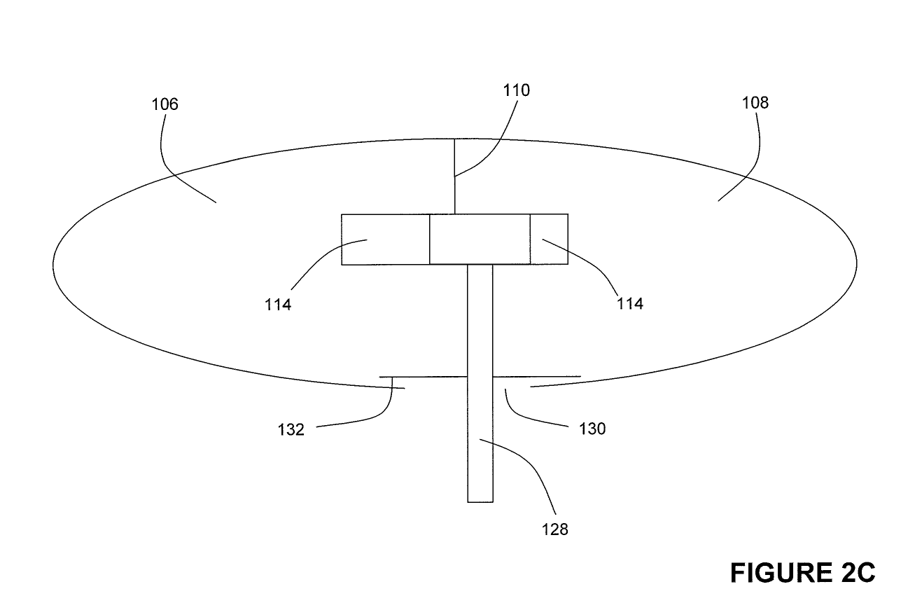

The first compartment 106 and the second compartment 108, while being generally separated by a barrier wall 110 therebetween, may both be in fluid communication with a mixing compartment 112, shown in FIG. 1, to be above the first compartment 106 and second compartment 108, through a common aperture 114 (best shown in FIGS. 2A-2C). A pump mechanism 116, such as the many pump mechanisms known by those skilled in the art for pumping ambient air, may be provided so as to pressurize the first compartment 106 and second compartment 108 with air pressure upon actuation of the pump. It will be appreciated that upon pressurization of the respective compartments 106, 108, portions of the first phase and second phase will be driven through the aperture 114 into the mixing compartment 112. Although not shown, the aperture 114 at each of the first compartment 106 and second compartment 108 may be provided with a flow inhibiting feature, such as a check valve (not shown), to prevent constituent that has flowed through the aperture 114 into the mixing chamber 112 from returning back to the respective compartment.

Once the portions of the first phase and second phase are pushed into the mixing compartment 112, the portions may be mixed together with an applicator 118. As generally known in the art, the applicator 118 may comprise a handle 120, shaft 122, and applicator portion 124, with the shaft connecting the handle to the applicator portion. The main body 102, and specifically the mixing compartment 112, may include an aperture 126 sized and configured to permit entry and exit of the applicator portion 124 of the applicator 118 along the path depicted by arrows B. The applicator portion 118 is preferably formed to a larger diameter than the shaft 122. By being so configured, it will be appreciated that upon entry of the entire applicator portion 124, the applicator 118 may be wiggled, twisted, or otherwise manipulated to fully mix the portions of the first phase and second phase found within the mixing chamber 112, as well as to sufficiently coat the applicator portion 124 with mixed phases in a satisfactory manner for use as a mascara.

Upon withdrawal of the applicator portion 124 of the applicator 118 from the mixing chamber 112, the cap 104 may be placed over the mixing chamber, in the direction of arrows C, to cover the aperture 126 and thereby seal the aperture to prevent the mixed phases from spilling out of the mixing chamber as well as to inhibit the infiltration of air into the mixing chamber such that the mixed phases remain as a liquid through a useful life of the inventive product. In other embodiments, the cap 104 may be configured to be placed over portions of the main body 102 and the applicator 118, such that the applicator may remain within the mixing chamber 112 with the cap in place.

Also shown in FIG. 1, and considered to be an optional feature of the product package 100, is a configuration lever 128. A purpose of the configuration lever 128 is to partially obstruct portions of the aperture 114 between the mixing compartment 112 and the first and second compartments 106, 108, such that the phases contained therein may be transferred to the mixing chamber 112 in weighted proportions.

Alternatively, as shown in FIGS. 2A-2C, if it is desired to permit the phases to enter the mixing chamber 112 in unequal proportions, the configuration lever 128 may be positioned accordingly within a configuration lever opening 130 formed within the main body 102 of the product package 100. For example, if it is desired to permit a greater amount of the phase in the second compartment 108 to enter the mixing chamber 112 than the amount of phase in the first compartment 106, the configuration lever may be moved to the position shown in FIG. 2A, such that a greater portion of the aperture 114 is revealed over the second compartment than the first compartment. Alternatively, if it is desired that a greater proportion of the first phase be permitted to enter the mixing chamber 112 than the second phase, the configuration lever 128 may be moved to the position shown in FIG. 2C, such that a greater portion of the aperture 114 is revealed over the first compartment 106 than the second compartment 108. One skilled in the art will appreciate that given equal pressure in the first compartment 106 and second compartment 108 (provided by the pump mechanism 116), and given equal viscosity of the two phases, the liquid phases will flow through a larger aperture more quickly than a smaller aperture. In this manner, the user may adjust the level of each phase permitted to enter the mixing chamber 112, and may thereby control product characteristics of the mascara product, as discussed more fully herein.

In order to block portions of the phases from being released through the configuration lever opening 130, the configuration lever may be provided with a blocking member 132 that moves along with the configuration lever while inhibiting the phases from passing. If provided with a configuration lever 128, it will be appreciated that the cap 104 may extend beyond the limits of the configuration lever opening 130, so as to inhibit air from entering or mascara product from exiting.

It has been surprisingly discovered that the use of a multi-phase mascara product it is possible to produce a mascara with a pleasant sensory experience and improved application properties while maintaining the desirable volume, length, texture, and wear properties of prior mascaras. Moreover, the multi-phase mascara product of the present invention allows the consumer to modify the properties of the mascara composition to suit her needs.

The following examples are intended to further illustrate the present invention. They are not intended to limit the invention in any way. Unless otherwise indicated, all parts are by weight.

### EXAMPLES

### Example 1

A mascara composition of the present invention is described below.

| | INGREDIENT | WT % |
|---|---|---|
| A1 | PEG 8 Beeswax | 22.00 |
| | Carnauba K82H | 5.50 |
| A2 | Methylparaben | 0.20 |
| A3 | PMMA | 2.00 |
| B | Carbon Black grind | 9.00 |
| | Water | 14.80 |
| C | Butylene Glycol | 5.00 |
| D | Sodium Polymethacrylate | 1.00 |
| E | PEG/PPG-17/18 | 2.00 |
| | Dimethicone | |
| F | Phenonip | 0.70 |
| G | NaOH (50% solution) | 0.20 |
| H | Water | 2.00 |
| | Polyquaternium-10 | 0.10 |
| I | Rayon Fibers | 0.44 |
| | Poly Toluylene Co | 0.06 |
| | Trimellic Amide Imide | |
| I1 | Diatosol 5000 SJ | 30.00 |
| I2 | Hybridur | 5.00 |
| | TOTAL | 100.00 |

The mascara composition described above was prepared as follows. The ingredients of Phase A1 were added to a beaker with heating at 85-90° C. The ingredients of Phases A2 and A3 were added and homogenized for 1 hour. The mixture was then placed in the first compartment of a product package.

The ingredients of Phases B though I2 were added sequentially to a second beaker with mixing until uniform. The mixture was then placed in the second compartment of a product package.

A portion of the mixture from the first compartment and a portion of the mixture from the second compartment are then pushed into a mixing compartment in the product package and mixed together with an applicator to form the mascara composition. The mascara composition is then applied to a keratinous tissue, such as eye lashes, with the applicator.

### Example 2

A mascara composition of the present invention is described below.

| | INGREDIENT | WT % |
|---|---|---|
| A1 | PEG 8 Beeswax | 22.00 |
| | Carnauba K82H | 5.50 |
| A2 | PMMA | 2.00 |
| B | Carbon Black grind | 12.00 |
| | Water | 18.60 |
| C | Butylene Glycol | 5.00 |
| | Methylparaben | 0.20 |
| | Ethylparaben | 0.20 |
| D | Sodium Polymethacrylate | 1.00 |
| E | Phenonip | 0.70 |
| F | NaOH (50% solution) | 0.20 |
| G | Water | 2.50 |
| | Polyquaternium-10 | 0.10 |
| H1 | Diatosol 5000 SJ | 25.00 |
| H2 | Hybridur | 5.00 |
| | TOTAL | 100.00 |

The mascara composition described above was prepared as follows. The ingredients of Phase A1 were added to a beaker with heating at 85-90° C. The ingredient of Phases A2 was added and homogenized for 1 hour. The mixture was then placed in the first compartment of a product package.

The ingredients of Phases B though H2 were added sequentially to a second beaker with mixing until uniform. The mixture was then placed in the second compartment of a product package.

A portion of the mixture from the first compartment and a portion of the mixture from the second compartment are then pushed into a mixing compartment in the product package and mixed together with an applicator to form the mascara composition. The mascara composition is then applied to a keratinous tissue, such as eye lashes, with the applicator.

### Example 3

A mascara composition of the present invention is described below.

| | INGREDIENT | WT % |
|---|---|---|
| A1 | PEG 8 Beeswax | 22.00 |
| | Carnauba K82H | 5.50 |
| A2 | PMMA | 2.00 |
| B | Carbon Black grind | 12.00 |
| | Water | 16.60 |
| C | Butylene Glycol | 5.00 |
| | Methylparaben | 0.20 |
| | Ethylparaben | 0.20 |
| D | Sodium Polymethacrylate | 1.00 |
| E | Phenonip | 0.70 |
| F | NaOH (50% solution) | 0.20 |
| G | Water | 2.50 |
| | Polyquaternium-10 | 0.10 |
| H1 | Diatosol 5000 SJ | 25.00 |
| H2 | Hybridur | 5.00 |
| I | Clear Mascara* | 2.00 |
| | TOTAL | 100.00 |

*Clear mascara is a combination of disodium EDTA (0.01-0.05%), triethanolamine (0.5-1.0%), phenoxyethanol (0.3-1.0%), methylparaben (0.2-0.4%), carbomer (0.1-0.9%), polyvinyl alcohol (0.1-0.8%), denatured ethanol (2-6%), glycerin (1-4%), propylene glycol (1-3%), panthenol (0.01-1%), and water (q.s.).

The mascara composition described above was prepared as follows. The ingredients of Phase A1 were added to a beaker with heating at 85-90° C. The ingredient of Phase A2 was added and homogenized for 1 hour. The mixture was then placed in the first compartment of a product package.

The ingredients of Phases B though I were added sequentially to a second beaker with mixing until uniform. The mixture was then placed in the second compartment of a product package.

A portion of the mixture from the first compartment and a portion of the mixture from the second compartment are then pushed into a mixing compartment in the product package and mixed together with an applicator to form the mascara composition. The mascara composition is then applied to a keratinous tissue, such as eye lashes, with the applicator.

### Example 4

A mascara composition of the present invention is described below.

| | INGREDIENT | WT % |
|---|---|---|
| A1 | PEG 8 Beeswax | 22.00 |
| | Carnauba K82H | 5.50 |
| A2 | PMMA | 2.00 |
| B | Carbon Black grind | 12.00 |
| | Water | 18.60 |
| C | Butylene Glycol | 5.00 |
| | Methylparaben | 0.20 |
| | Ethylparaben | 0.20 |
| D | Sodium Polymethacrylate | 1.00 |
| E | Phenonip | 0.70 |
| F | NaOH (50% solution) | 0.20 |
| G | Water | 2.50 |
| | Polyquaternium-10 | 0.10 |
| H1 | Diatosol 5000 SJ | 25.00 |
| H2 | Hybridur | 5.00 |
| | TOTAL | 100.00 |

The mascara composition described above was prepared as follows. The ingredients of Phase A1 were added to a beaker with heating at 85-90° C. The ingredient of Phase A2 was added and homogenized for 1 hour. The mixture was then placed in the first compartment of a product package.

The ingredients of Phases B though H2 were added sequentially to a second beaker with mixing until uniform. The mixture was then placed in the second compartment of a product package.

A portion of the mixture from the first compartment and a portion of the mixture from the second compartment are then pushed into a mixing compartment in the product package and mixed together with an applicator to form the mascara composition. The mascara composition is then applied to a keratinous tissue, such as eye lashes, with the applicator.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A multi-phase mascara product, comprising a first container having disposed therein a first phase comprising a wax, a second container having disposed therein a second phase comprising a film forming polymer, and a third container into which relative amounts of said first and second phases may be introduced, wherein upon mixing of the first and second phases, the third container comprises the wax, the film-forming polymer, a solvent and a thickener, wherein if the solvent and thickener are oil-soluble, they are present in the first phase, and if the solvent is aqueous and the thickener is water-soluble, they are present in the second phase.

2. The mascara product of claim 1, wherein said solvent comprises water.

3. The mascara product of claim 2, wherein said first phase further comprises an emulsifier.

4. The mascara product of claim 1, wherein said solvent comprises a hydrocarbon solvent.

5. The mascara product of claim 1, wherein said wax comprises beeswax.

6. The mascara product of claim 1, wherein said wax comprises carnauba wax.

7. The mascara product of claim 1, wherein said second phase comprises said film forming polymer.

8. The mascara product of claim 7, wherein said film forming polymer comprises an acrylates copolymer.

9. The mascara product of claim 7, wherein said film forming polymer comprises a polyurethane/poly(meth)acrylate graft copolymer.

10. The mascara product of claim 7, wherein said second phase further comprises iridescent particles.

11. The mascara product of claim 7, wherein said first phase is free of a film forming polymer.

12. The mascara product of claim 7, wherein said first phase further comprises a film forming polymer which may be the same as or different from said film forming polymer in said second phase.

13. The mascara product of claim 7, wherein said first phase further comprises a thickener.

14. The mascara product of claim 7, wherein said first phase further comprises fibers.

15. The mascara product of claim 7, wherein said second phase is free of said thickener or said fibers.

16. The mascara product of claim 1, wherein said second phase comprises said fibers.

17. The mascara product of claim 16, wherein said first phase is free of fibers.

18. The mascara product of claim 16, wherein said first phase further comprises a film forming polymer and a thickener.

19. The mascara product of claim 16, wherein said second phase is free of said film forming polymer and said thickener.

20. The mascara product of claim 1, wherein said second phase comprises said thickener.

21. The mascara product of claim 20, wherein said first phase is free of thickener.

22. The mascara product of claim 20, wherein said second phase is free of said film forming polymer and said fibers.

23. The mascara product of claim 20, wherein said first phase further comprises a film forming polymer.

24. The mascara product of claim 20, wherein said second phase further comprises mothers of pearl.

25. The mascara product of claim 1, wherein said first phase further comprises a colorant.

26. The mascara product of claim 25, wherein said colorant comprises reflective particles, and wherein said film forming polymer in said second phase exhibits high gloss or wherein said second phase further comprises a high gloss oil.

27. The mascara product of claim 25, wherein said second phase further comprises a colorant which is different from said colorant in said first phase.

28. A process for making a mascara, comprising:
a) providing a multi-phase mascara product, comprising a first container having disposed therein a first phase comprising a wax, a second container having disposed therein a second phase comprising a film forming polymer, and a third container into which relative amounts of said first and second phases may be introduced, wherein upon mixing of the first and second phases, the third container comprises the wax, the film-forming polymer, a solvent and a thickener, wherein if the solvent and thickener are oil-soluble, they are present in the first phase, and if the solvent is aqueous and the thickener is water-soluble, they are present in the second phase,
b) introducing the relative amounts of said first and second phases into said third container;
c) mixing together the relative amounts said first and second phases to form a mascara.

29. A method of making up or enhancing the appearance of eye lashes comprising applying to the eye lashes a mascara made by the process of claim 28.

30. A multi-phase mascara product, comprising:
a) a main body comprising a first container, a second container, and a third container, wherein the first and second containers are each in fluid communication with the third container,
b) the first container having disposed therein a first phase comprising a wax, and
c) the second container having disposed therein a second phase comprising a film forming polymer,
wherein relative amounts of said first and second phases may be introduced into and mixed in the third container, wherein upon mixing of the first and second phases, the third container comprises the wax, the film-forming polymer, a solvent and a thickener, wherein if the solvent and thickener are oil-soluble, they are present in the first phase, and if the solvent is aqueous and the thickener is water-soluble, they are present in the second phase.

31. The mascara product of claim 30, wherein the first and second containers are in fluid communication with the third container through a common aperture.

32. The mascara product of claim 31, further comprising a pump mechanism, which when actuated pressurizes the first container, the second container, or both and forces a portion of the first phase, the second phase, or both, respectively, through the aperture and into the third container.

33. The mascara product of claim 30, further comprising an applicator, and wherein the third container comprising an aperture sized and configured to permit entry and exit of at least a portion of the applicator.

34. The mascara product of claim 32, further comprising a movable configuration lever partially obstructing the aperture, such that the amount of the first phase, the second phase, or both that is forced through the aperture and into the third container upon actuation of the pump mechanism may be controlled by the position of the configuration lever.
